(19) United States
(12) Patent Application Publication
(10) Pub. No.: US 2005/0202030 A1
(43) Pub. Date: Sep. 15, 2005

(54) Method of evaluating the therapeutic potential of a vaccine for mucosal administration

(75) Inventors: Hans-Henrik Ipsen, Hillerod (DK); Lise Lund Maerkedahl, Fredensborg (DK); Henrik H. Jacobi, Vedbaek (DK)

Correspondence Address: DARBY & DARBY P.C., P. O. BOX 5257, NEW YORK, NY, 10150-5257, US

(73) Assignee: ALK-Abello A/S, Horsholm (DK)

(21) Appl. No.: 11/068,718

(22) Filed: Feb. 23, 2005

Related U.S. Application Data
(60) Provisional application No. 60/548,454, filed on Feb. 26, 2004.
(60) Provisional application No. 60/559,095, filed on Apr. 2, 2004.

(30) Foreign Application Priority Data
Feb. 26, 2004 (DK) PA 2004 00310

(51) Int. Cl. G01N 33/53; A61K 39/00

(52) U.S. Cl. 424/184.1; 435/7.1

(57) ABSTRACT

The invention relates to a method of evaluating the therapeutic potential of a vaccination program comprising a vaccine for mucosal administration comprising one or more antigens and a vaccination protocol, the method comprising a) subjecting at least one test individual to the vaccination program, b) measuring the level of a biomarker antibody selected from the group consisting of IgA, IgG, IgE and IgX specific to the antigen in a biological sample from the test individual, and c) using the measurements obtained to evaluate the therapeutic potential of the vaccination program.

### TECHNICAL FIELD

The present invention relates to a method of evaluating the therapeutic potential of a vaccine for mucosal administration comprising one or more antigens.

### BACKGROUND OF THE INVENTION

Allergy is a major health problem in countries where Western lifestyle is adapted. Furthermore, the prevalence of allergic disease is increasing in these countries. Although allergy in general may not be considered a life-threatening disease, asthma annually causes a significant number of deaths. An exceptional prevalence of about 30% in teenagers conveys a substantial loss in quality of life, working days and money, and warrants a classification among major health problems in the Western world.

Allergy is a complex disease. Many factors contribute to the sensitisation event. Among these is the susceptibility of the individual defined by an as yet insufficiently understood interplay between several genes. Another important factor is allergen exposure above certain thresholds. Several environmental factors may be important in the sensitisation process including pollution, childhood infections, parasite infections, intestinal microorganisms, etc. Once an individual is sensitised and the allergic immune response established, the presence of only minute amounts of allergen is efficiently translated into symptoms.

The natural course of allergic disease is usually accompanied by aggravation at two levels. Firstly, a progression of symptoms and disease severity, as well as disease progression, for example from hay fever to asthma. Secondly, dissemination in offending allergens most often occurs resulting in allergic multi-reactivity. Chronic inflammation leads to a general weakening of the mucosal defense mechanisms resulting in unspecific irritation and eventually destruction of the mucosal tissue. Infants may become sensitised primarily to foods, i.e. milk, resulting in eczema or gastrointestinal disorders; however, most often they outgrow these symptoms spontaneously. These infants are at risk of developing inhalation allergy later in their lives.

The most important allergen sources are found among the most prevalent particles of a certain size in the air we breathe. These sources are remarkably universal and include grass pollens and house dust mite faecal particles, which together are responsible for approximately 50% of all allergies. Of global importance are also animal dander, i.e. cat and dog dander, other pollens, such as mugwort pollens, and micro-fungi, such as Alternaria. On a regional basis yet other pollens may dominate, such as birch pollen in Northern and Central Europe, ragweed in the Eastern and Central United States, and Japanese cedar pollen in Japan. Insects, i.e. bee and wasp venoms, and foods each account for approximately 2% of all allergies.

Allergy, i.e. type I hyper-sensitivity, is caused by an inappropriate immunological reaction to foreign non-pathogenic substances. Important clinical manifestations of allergy include asthma, hay fever, eczema, and gastro intestinal disorders. The allergic reaction is specific in the sense that a particular individual is sensitised to particular allergen(s), whereas the individual does not necessarily show an allergic reaction to other substances known to cause allergic disease. The allergic phenotype is characterized by a pronounced inflammation of the mucosa of the target organ and by the presence of allergen specific antibody of the IgE class in the circulation and on the surfaced of mast-cells and basophils.

An allergic attack is initiated by the reaction of the foreign allergen with allergen specific IgE antibodies, when the antibodies are bound to high affinity IgE specific receptors on the surface of mast-cells and basophils. The mast-cells and basophils contain preformed mediators, i.e. histamine, tryptase, and other substances, which are released upon cross-linking of two or more receptor-bound IgE antibodies. IgE antibodies are cross-linked by the simultaneous binding of one allergen molecule. The cross-linking of receptor bound IgE on the surface of mast-cells also leads to release of signaling molecules responsible for the attraction of eosinophils, allergen specific T-cells, and other types of cells to the site of the allergic response. These cells in interplay with allergen, IgE and effector cells, lead to a renewed flash of symptoms occurring 12-24 hours after allergen encounter (late phase reaction).

Allergy disease management comprises diagnosis and treatment including prophylactic treatments. Diagnosis of allergy is concerned with the demonstration of allergen specific IgE and identification of the allergen source. In many cases a careful anamnesis may be sufficient for the diagnosis of allergy and for the identification of the offending allergen source material. Most often, however, the diagnosis is supported by objective measures, such as skin prick test, blood test, or provocation test.

The therapeutic options fall in three major categories. The first opportunity is allergen avoidance or reduction of the exposure. Whereas allergen avoidance is obvious e.g. in the case of food allergens, it may be difficult or expensive, as for house dust mite allergens, or it may be impossible, as for pollen allergens. The second and most widely used therapeutic option is the prescription of classical symptomatic drugs like anti-histamines and steroids. Symptomatic drugs are safe and efficient; however, they do not alter the natural cause of the disease, neither do they control the disease dissemination. The third therapeutic alternative is specific allergy vaccination that in most cases reduces or alleviates the allergic symptoms caused by the allergen in question.

Conventional specific allergy vaccination is a causal treatment for allergic disease. It interferes with basic immunological mechanisms resulting in persistent improvement of the patients' immune status. Thus, the protective effect of specific allergy vaccination extends beyond the treatment period in contrast to symptomatic drug treatment. Some patients receiving the treatment are cured, and in addition, most patients experience a relief in disease severity and symptoms experienced, or at least an arrest in disease aggravation. Thus, specific allergy vaccination has preventive effects reducing the risk of hay fever developing into asthma, and reducing the risk of developing new sensitivities.

The immunological mechanism underlying successful allergy vaccination is not known in detail. A specific immune response, such as the production of antibodies against a particular pathogen, is known as an adaptive immune response. This response can be distinguished from the innate immune response, which is an unspecific reaction towards pathogens. An allergy vaccine is bound to address the adaptive immune response, which includes cells and molecules with antigen specificity, such as T-cells and the antibody producing B-cells. B-cells cannot mature into antibody producing cells without help from T-cells of the corresponding specificity. T-cells that participate in the stimulation of allergic immune responses are primarily of the Th2 type. Establishment of a new balance between Th1 and Th2 cells has been proposed to be beneficial and central to the immunological mechanism of specific allergy vaccination. Whether this is brought about by a reduction in Th2 cells, a shift from Th2 to Th1 cells, or an up-regulation of Th1 cells is controversial. Recently, regulatory T-cells have been proposed to be important for the mechanism of allergy vaccination. According to this model regulatory T-cells, i.e. Th3 or Tr1 cells, down-regulate both Th1 and Th2 cells of the corresponding antigen specificity. In spite of these ambiguities it is generally believed that an active vaccine must have the capacity to stimulate allergen specific T-cells.

Specific allergy vaccination is, in spite of its virtues, not in widespread use, primarily for two reasons. One reason is the inconveniences associated with the traditional vaccination programme that comprises repeated vaccinations i.a. injections over a several months. The other reason is, more importantly, the risk of allergic side reactions. Ordinary vaccinations against infectious agents are efficiently performed using a single or a few high dose immunizations. This strategy, however, cannot be used for allergy vaccination since a pathological immune response is already ongoing.

Conventional specific allergy vaccination is therefore carried out using multiple subcutaneous immunizations applied over an extended time period. The course is divided in two phases, the up dosing and the maintenance phase. In the up dosing phase increasing doses are applied, typically over a 16-week period, starting with minute doses. When the recommended maintenance dose is reached, this dose is applied for the maintenance phase, typically with injections every six weeks. Following each injection the patient must remain under medical attendance for 30 minutes due to the risk of anaphylactic side reactions, which in principle although extremely rare could be life-threatening. In addition, the clinic should be equipped to support emergency treatment. There is no doubt that a vaccine based on a different route of administration would eliminate or reduce the risk for allergic side reactions inherent in the current subcutaneous based vaccine as well as would facilitate a more widespread use, possibly even enabling self vaccination at home.

Attempts to improve vaccines for specific allergy vaccination have been performed for over 30 years and include multifarious approaches. Several approaches have addressed the allergen itself through modification of the IgE reactivity. Other approaches have addressed the formulation and administration route for the vaccine.

The testing of new allergens, allergen formulations and treatment protocols is both laborious and work- and time-consuming, since it requires in vivo testing in animals and/or humans, including clinical trials. In particular, clinical trials for testing the therapeutic efficacy of new vaccines or treatment protocols are work- and time consuming, since they traditionally involve a high number of patients, a long treatment period, which for example for pollen allergy comprises the pollen season and a period prior to the pollen season, and a broad range of doses. Moreover, clinical trials require governmental approval of the clinical study to be carried out and enrolment of suitable test persons. Also, clinical trials of vaccines, e.g. allergy vaccines, rely on the monitoring and evaluation of clinical symptoms, which are highly subjective parameters, and hence the results of such trials involves a certain degree of uncertainty.

The object of the present invention is to provide an improved method of evaluating the therapeutic potential of a vaccination protocol or a vaccine for mucosal administration, e.g. a vaccine for oromucosal, i.a. sublingual, administration.

### SUMMARY OF THE INVENTION

This object is achieved by the present invention, which comprises i.a. the following aspects:

A method of evaluating the therapeutic potential of a vaccination program comprising a vaccine for mucosal administration comprising one or more antigens and a vaccination protocol, the method comprising
a) subjecting at least one test individual to the vaccination program,
b) measuring the level of a biomarker antibody selected from the group consisting of IgA, IgG, IgE and IgX specific to the antigen in a biological sample from the test individual, and
c) using the measurements obtained to evaluate the therapeutic potential of the vaccination program.

A vaccine obtainable by the method according to any of claims 1-35.

A method of evaluating the effect of mucosal Specific Allergy Vaccination (SAV) on an individual, the method comprising
a) subjecting the individual to mucosal SAV,
b) measuring the level of a biomarker antibody selected from the group consisting of IgA, IgG, IgE and IgX specific to the antigen in a biological sample from the individual, and
c) using the measurements obtained to evaluate the effect of the SAV.

The invention is based on the surprising experimental finding that mucosal administration of an allergy vaccine is followed by a distinct and reliable increase in the blood fluid concentration of both IgE, IgG, IgA and IgX specific to the allergen in question, whereas the mucosal administration of a vaccine does not give rise to any changes for a number of other measurable markers of the response of the immune system. The experimental results suggest that body fluid concentrations of antigen-specific IgE, IgG, IgA and IgX are strong and effective markers for the therapeutic effect of a vaccine. Thus, the present invention provides a possibility of testing new allergen candidates, new vaccine formulations as well as new vaccination protocols by in vitro assaying of a biological sample from a test animal or individual. By e.g. carrying out such in vitro assaying prior to clinical trials it is possible to exclude non-effective vaccination protocols, vaccines and doses before embarking on clinical trials and hence to reduce the extent of clinical trials thereby making the development of new vaccines more feasible.

Also, the invention has provided a new and additional parameter of evaluating the therapeutic potential of a vaccine or vaccination protocol, a parameter which is further easily measurable and quantifiable and hence very reliable. In particular, the method may be used in connection with clinical trials to obtain additional information or to obtain information alternative to other parameters, such as clinical symptoms, and thereby making clinical trial more reliable.

As will appear from the above, the method may be used in any stage of vaccine development from screening of new allergen candidates, e.g. recombinant and recombinant modified allergens, to clinical trials of vaccines for the purpose of obtaining market authorisations of vaccines.

### SHORT DESRIPTION OF THE FIGURES

FIG. 1 shows the IgE level for a group of patients treated with placebo for four points in time of the administration period.

FIG. 2 shows the IgE level for a group of patients treated with a dose of 2500 SQ for four points in time of the administration period.

FIG. 3 shows the IgE level for a group of patients treated with a dose of 25000 SQ for four points in time of the administration period.

FIG. 4 shows the IgE level for a group of patients treated with a dose of 75000 SQ for four points in time of the administration period.

FIG. 5 shows the IgA level for a group of patients treated with placebo for four points in time of the administration period.

FIG. 6 shows the IgA level for a group of patients treated with a dose of 2500 SQ for four points in time of the administration period.

FIG. 7 shows the IgA level for a group of patients treated with a dose of 25000 SQ for four points in time of the administration period.

FIG. 8 shows the IgA level for a group of patients treated with a dose of 75000 SQ for four points in time of the administration period.

FIG. 9 shows the IgG level for a group of patients treated with placebo for four points in time of the administration period.

FIG. 10 shows the IgG level for a group of patients treated with a dose of 2500 SQ for four points in time of the administration period.

FIG. 11 shows the IgG level for a group of patients treated with a dose of 25000 SQ for four points in time of the administration period.

FIG. 12 shows the IgG level for a group of patients treated with a dose of 75000 SQ for four points in time of the administration period.

FIG. 13 shows the IgE level for a group of patients treated with placebo at the start of and at 1-2 weeks after a treatment period.

FIG. 14 shows the IgE level for a group of patients treated with a dose of 1000000 SQ at the start of and at 1-2 weeks after a treatment period.

FIG. 15 shows the IgE level for Treatments Groups 1-4 of the Intention-To-Treat (ITT) population at three points of time during the treatment period.

FIG. 16 shows the IgE level for Treatments Groups 1, 5 and 6 of the Intention-To-Treat (ITT) population at three points of time during the treatment period.

FIG. 17 shows the IgE level for Treatments Groups 1-4 of the Per-Protocol (PP) population at three points of time during the treatment period.

FIG. 18 shows the IgE level for Treatments Groups 1, 5 and 6 of the Per-protocol (PP) population at three points of time during the treatment period.

FIG. 19 shows the IgX level for Treatments Groups 1-4 of the Intention-To-Treat (ITT) population at three points of time during the treatment period.

FIG. 20 shows the IgX level for Treatments Groups 1, 5 and 6 of the Intention-To-Treat (ITT) population at three points of time during the treatment period.

FIG. 21 shows the IgX level for Treatments Groups 1-4 of the Per-Protocol (PP) population at three points of time during the treatment period.

FIG. 22 shows the IgX level for Treatments Groups 1, 5 and 6 of the Per-Protocol (PP) population at three points of time during the treatment period.

### DETAILED DESCRIPTION OF THE INVENTION

Evaluation of the Therapeutic Potential of a Vaccine Program

The evaluation of the therapeutic potential of a vaccination program may be based on 1) measurements of IgE, IgA, IgG or IgX alone, 2) measurements of two of the four biomarker antibodies, 3) measurements of three of the four biomarker antibodies, or 4) measurements of all four biomarker antibodies. The evaluation of the measurements obtained may be carried out by comparison with a reference temporal biomarker antibody level profile, e.g. by preparing a plot of the biomarker antibody level versus time to produce a temporal curve. The measurements obtained is used to evaluate the therapeutic potential of a given vaccination program to be tested. The vaccination program has two principal components, viz. the vaccination protocol and the vaccine, which each has a number of parameters, which may be the subject of testing in the method of the invention. The vaccination protocol involves the parameters of e.g. the screening and selection of persons for the study, the circumstances under which administration of the vaccine takes place, the route of administration, the doses of antigen used, the frequency and duration of administration etc. The vaccine involves the parameters of one or more antigens, the adjuvant, if present, and the formulation of the vaccine, i.e. the excipients of the formulation.

The method of the invention may be used to evaluate the therapeutic potential of 1) the vaccination protocol using a conventional vaccine, 2) the vaccine using a conventional vaccination protocol or 3) the combination of the vaccination protocol and the vaccine.

In one embodiment of the method of the invention, the biomarker antibody is IgE. It is preferred that the IgE level increases more than 50%, preferably more than 100%, more preferably more than 200%, more preferably more than 300% and more preferably more than 400% compared to the level at the start of the vaccination program. It is further preferred that the IgE level increase occurs within twelve weeks, preferably within ten weeks, more preferably within eight weeks, more preferably within six weeks, and most preferably within four weeks from the start of the vaccination program.

In a particular embodiment of the invention, the IgE level has a maximum value followed by a decrease. It is preferred that the maximum value of the IgE level occurs within twelve weeks, preferably within ten weeks, more preferably within eight weeks, more preferably within six weeks, and more preferably within four weeks from the start of the vaccination program. It is preferred that the IgE level decreases to a level of below 90%, preferably below 80%, more preferably below 70%, more preferably below 60%, and most preferably below 50% of the maximum value. It is preferred that the IgE level decrease occurs within 26 weeks, preferably within 20 weeks, more preferably within 16 weeks, more preferably within 12 weeks, more preferably within 8 weeks, and most preferably within 4 weeks from the time of the maximum value.

In another embodiment of the invention, the IgE level remains at approximately the optimum value once it has been reached.

In a further preferred embodiment of the invention, the biomarker antibody is IgA. It is preferred that the IgA level increases more than 50%, preferably more than 100%, more preferably more than 200%, more preferably more than 300% and most preferably more than 400% compared to the level at the start of the vaccination program. It is preferred that the IgA level increase occurs within 20 weeks, preferably within 16 weeks, more preferably within 12 weeks and most preferably within 8 weeks from the start of administration.

In yet a further preferred embodiment of the invention, the biomarker antibody is IgG. It is preferred that the IgG level increases more than 50%, preferably more than 100%, more preferably more than 200%, more preferably more than 300% and most preferably more than 400% compared to the level at the start of the vaccination program. It is preferred that the IgG level increase occurs within 20 weeks, preferably within 16 weeks, more preferably within 12 weeks and most preferably within 8 weeks from the start of administration.

In a further preferred embodiment of the invention the biomarker antibody is IgX. In a preferred embodiment of the invention, the IgX is expressed as the ratio of the level of IgE as measured in an immunoassay with competition from other components of the biological sample to the level of IgE as measured in an immunoassay with no competition. In this embodiment of the invention, it is preferred that the level of IgX decreases more than 4%, preferably more than 6%, more preferably more than 8%, more preferably more than 10%, more preferably more than 12% and most preferably more than 14% compared to the level at the start of the vaccination program. It is further preferred that the IgX level decrease occurs within 24 weeks, preferably within 16 weeks, more preferably within 12 weeks and most preferably within 8 weeks from the start of administration.

The expressions “IgE level”, “IgA level”, “IgG level” and “IgX level” used above may refer to the levels of single test individuals, or, when a group of individuals are subjected to the vaccination program, the weighted levels of the group or part of the group, such as the average levels thereof. Often the level of IgA, IgE, IgG and IgX varies from one individual to another, and therefore it is preferred to use a group of two or more, preferably five or more, more preferably ten or more individuals for the testing of a vaccination program.

Vaccination Protocol

In one embodiment of the invention, the vaccination protocol comprises daily administration of the vaccine to the test individual. In another embodiment of the invention the vaccination protocol comprises administration of the vaccine every second day, every third day or every fourth day. For instance, the vaccination protocol comprises administration of the vaccine for a period of more than 4 weeks, preferably more than 8 weeks, more preferably more than 12 weeks, more preferably more than 16 weeks, more preferably more than 20 weeks, more preferably more than 24 weeks, more preferably more than 30 and most preferably more than 36 weeks.

The period of administration may a continuous period. Alternatively, the period of administration is a discontinuous period interrupted by one or more periods of non-administration. Preferably, the (total) period of non-administration is shorter than the (total) period of administration.

In a further embodiment of the invention, the vaccine is administered to the test individual once a day. Alternatively, the vaccine is administered to the test individual twice a day. The vaccine may be a uni-dose vaccine.

Measurement of IgE, IgA, IgG and IgX

In one embodiment of the invention, the measuring of the level of IgA, IgE and/or IgG specific to the antigen in a biological sample is carried out by an ELISA comprising the steps of 1) coating the allergen onto an Elisa plate and washing, 2) adding the biological sample, incubating and washing, 3) adding a conjugate of an enzyme and anti-IgA/IgG/IgE antibody, incubating and washing, 4) adding an enzyme substrate, incubating and stopping the reaction, and 5) measuring the level of reacted substrate.

In another embodiment of the invention, the measuring of the level of IgA, IgE and/or IgG specific to the antigen in a biological sample is carried out by a two-site immunoassay (competition immunoassay) comprising the steps of
(a) providing a mixture of a liquid phase and a four-component solid phase complex composed of (i) the antibody of the sample, (ii) a class-specific antibody directed against the antibody to be detected bound to a solid phase, (iii) a ligand in the form of an antigen, an antibody or a hapten, and (iv) a label compound, to form a four-component solid phase complex,
(b) separating the four-component solid phase complex from the liquid phase,
(c) washing the separated four-component solid phase to remove non-complex bound compounds,
(d) performing a detection/measurement of the washed labelled four-component complex.

The two-site immunoassay may be carried out in a number of variant procedures. Optionally, a washing step may be carried out after mixing reagents (i) and (ii) before adding reagents (iii) and (iv) and/or after mixing reagents (i), (ii) and (iii). The solid phase may e.g. be a particulate carrier, such as paramagnetic particles. The label compound may e.g. be a chemiluminescent compound, such as an acridinium ester. The ligand may be bound to biotin or a functional derivative thereof, and the label compound may be a chemiluminescent compound covalently bound to avidin, streptavidin or a functional derivative thereof, in which case the detection/measurement of the washed labelled four-component complex is carried out by initiating a chemiluminescent reaction in the complex and detecting/measuring the resulting chemiluminescense, if any. The immunoassay may be carried out using e.g. an ADVIA Centaur (Bayer).

A particular embodiment of the two-site immunoassay (no competition immunoassay) comprises the steps of
(a) mixing (i) the biological sample with (ii) a class-specific antibody directed against the antibody to be detected bound to a solid phase to form a mixture of a liquid phase and a two-component solid phase complex,
(b) separating the two-component solid phase complex from the liquid phase and washing the separated two-component solid phase complex to remove non-complex bound compounds,
(c) adding (iii) a ligand in the form of an antigen, an antibody or a hapten, and (iv) a label compound, to form a four-component solid phase complex,
(d) separating the four-component solid phase complex from the liquid phase,
(e) washing the separated four-component solid phase to remove non-complex bound compounds,
(f) performing a detection/measurement of the washed labelled four-component complex.

Another particular embodiment of the two-site immunoassay (competition immunoassay) comprises a two-site immunoassay for an antibody using a chemiluminescent label and a biotin bound ligand, said method comprising the steps of
(a) mixing the biological sample with a ligand antigen, antibody or hapten bound to biotin or a functional derivative thereof, a class-specific antibody directed against the antibody to be detected bound to paramagnetic particles and a chemiluminescent acridinium compound bound to avidin, streptavidin or a functional derivative thereof to form a four-component solid phase complex,
(b) magnetically separating the four-component solid phase from the liquid phase,
(c) washing the separated four-component solid phase to remove non-complex bound compounds,
(d) initiating a chemiluminescent reaction, if any, in the separated solid phase and detecting/measuring the resulting chemiluminescence, if any.

A further particular embodiment of the two-site immunoassay (no competition immunoassay) comprises the steps of
(a) mixing (i) the biological sample with (ii) a class-specific antibody directed against the antibody to be detected bound to paramagnetic particles to form a mixture of a liquid phase and a two-component solid phase complex,
(b) magnetically separating the two-component solid phase complex from the liquid phase and washing the separated two-component solid phase complex to remove non-complex bound compounds,
(c) adding (iii) a ligand in the form of an antigen, an antibody or a hapten bound to biotin or a functional derivative thereof, and (iv) a chemiluminescent acridinium compound bound to avidin, streptavidin or a functional derivative thereof, to form a four-component solid phase complex,
(d) magnetically separating the four-component solid phase complex from the liquid phase,
(e) washing the separated four-component solid phase to remove non-complex bound compounds,
(f) initiating a chemiluminescent reaction, if any, in the washed solid phase, and detecting/measuring the resulting chemiluminescence, if any.

Preferably, IgX is expressed as the ratio of the level of IgE as measured in an immunoassay with competition from other components of the biological sample to the level of IgE as measured in an immunoassay with no competition. In this case the immunoassay with no competition may be one of the two-site no competition immunoassay embodiments as described above, wherein a washing step is carried out after mixing of the biological sample and the class-specific antibody directed against the antibody to be detected bound to a solid phase, i.e. washing is carried out before addition of the allergen in order to avoid competition from non-IgE antibodies of the biological sample in the binding of IgE antibodies of the sample to the allergen. The immunoassay with competition may be one of the two-site competition immunoassay embodiments mentioned above.

Preferably, the biological sample is selected from the group consisting of blood, plasma, serum, urine, saliva and nasal secretion.

Vaccine Formulations and Routes of Administration

In a preferred embodiment of the invention, the vaccine is selected from the group consisting of vaccines formulated so as to be adapted to administration via the oromucosa, the mucosa of the respiratory system, the mucosa of the digestive system, the rectal mucosa and the genital mucosa. Preferably, the vaccine is formulated so as to be adapted to administration via the oromucosa.

In a particular embodiment of the invention, the vaccine is selected from the group consisting of vaccines formulated as a solution, a suspension, a dispersion, an emulsion, fast dispersing dosage forms, drops, lozenges, a spray, an aerosol, a tablet, a chewable tablet, granules, a powder, a gel, a paste, a syrup, a cream, an ointment, a stick, implants, vagitories, suppositories or uteritories. Preferably, the vaccine is formulated as a fast dispersing dosage form. The formulation may be any conventional formulation suitable for mucosal administration, and in particular the formulation may comprise conventional excipients and adjuvants.

Antigens

The antigen of the vaccine evaluated according to the method of the present invention may be any antigen eliciting an immune response upon exposure to an individual. In a preferred embodiment of the invention, the antigen is selected from the group consisting of a respiratory antigen, a digestive antigen, a microbial antigen and an insect antigen. In a particular embodiment of the invention, the antigen is a respiratory antigen.

In a particular embodiment, the antigen is an allergen, e.g. an inhalant allergen or an insect allergen. Other examples of antigens are allergoids, peptides, haptens, carbohydrates, peptide nucleic acids (PNAs, a sort of synthetic genetic mimic), and infectious antigens, such as viral or bacterial material, as well as analogues or derivatives thereof. Examples of nutritional substances are vitamins, enzymes, trace elements, and trace minerals as well as analogues or derivatives thereof. Examples of medicaments are antibodies, antibiotics, peptides, salts, hormones, hemolytics, hemostatics, enzymes, enzyme inhibitors, psycopharmica, opiates, and barbiturates, as well as analogues or derivatives thereof.

Allergens

In a preferred embodiment of the invention the allergen is any naturally occurring protein that has been reported to induce allergic, i.e. IgE mediated reactions upon their repeated exposure to an individual. Examples of naturally occurring allergens include pollen allergens (tree-, herb, weed-, and grass pollen allergens), insect allergens (inhalant, saliva and venom allergens, e.g. mite allergens, cockroach and midges allergens, hymenopthera venom allergens), animal hair and dandruff allergens (from e.g. dog, cat, horse, rat, mouse etc.), and food allergens. Important pollen allergens from trees, grasses and herbs are such originating from the taxonomic orders of Fagales, Oleales, Pinales and platanaceae including i.a. birch (Betula), alder (Alnus), hazel (Corylus), hornbeam (Carpinus) and olive (Olea), cedar (Cryptomeria and Juniperus), Plane tree (Platanus), the order of Poales including i.a. grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other important inhalation allergens are those from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (superfamily Apidae), wasps (superfamily Vespidea), and ants (superfamily Formicoidae). Important inhalation allergens from fungi are i.a. such originating from the genera Alternaria and Cladosporium.

In a more preferred embodiment of the invention the allergen is Bet v 1, Aln g 1, Cor a 1 and Car b 1, Que a 1, Cry j 1, Cry j 2, Cup a 1, Cup s 1, Jun a 1, Jun a 2, jun a 3, Ole e 1, Lig v1, Pla l 1, Pla a 2, Amb a 1, Amb a 2, Amb t 5, Art v 1, Art v 2 Par j 1, Par j 2, Par j 3, Sal k 1, Ave e 1, Cyn d 1, Cyn d 7, Dac g 1, Fes p 1, Hol l 1, Lol p 1 and 5, Pha a 1, Pas n 1, Phl p 1, Phl p 5, Phl p6, Poa p 1, Poa p 5, Sec c1, Sec c 5, Sor h 1, Der f 1, Der f 2, Der p 1, Der p 2, Der p 7, Der m 1, Eur m 2, Gly d 1, Lep d 2, Blot t 1, Tyr p 2, Bla g 1, Bla g 2, Per a 1, Fel d 1, Can f 1, Can f 2, Bos d 2, Equ c 1, Equ c 2, Equ c 3, Mus m 1, Rat n 1, Apis m 1, Api m 2, Ves v 1, Ves v 2, Ves v 5, Dol m 1, Dil m 2, Dol m 5, Pol a 1, Pol a 2, Pol a 5, Sol i 1, Sol i 2, Sol i 3 and Sol i 4, Alt a 1, Cia h 1, Asp f 1, Bos d 4, Mal d 1, Gly m 1, Gly m 2, Gly m 3, Ara h 1, Ara h 2, Ara h 3, Ara h 4, Ara h 5 or shufflant hybrids from Molecular Breeding of any of these.

In the most preferred embodiment of the invention the allergen is grass pollen allergen or a dust mite allergen or a ragweed allergen or a cedar pollen or a cat allergen or birch allergen.

In yet another embodiment of the invention the fast dispersing solid dosage form comprises at least two different types of allergens either originating from the same allergic source or originating from different allergenic sources e.g. grass group 1 and grass group 5 allergens or mite group 1 and group 2 allergens from different mite and grass species respectively, weed antigens like short and giant ragweed allergens, different fungis allergens like alternaria and cladosporium, tree allergens like birch, hazel, hornbeam, oak and alder allergens, food allergens like peanut, soybean and milk allergens.

The allergen incorporated into the fast dispersing solid dosage form may be in the form of an extract, a purified allergen, a modified allergen, a recombinant allergen or a mutant of a recombinant allergen. An allergenic extract may naturally contain one or more isoforms of the same allergen, whereas a recombinant allergen typically only represents one isoform of an allergen. In a preferred embodiment the allergen is in the form of an extract. In another preferred embodiment the allergen is a recombinant allergen. In a further preferred embodiment the allergen is a naturally occurring low IgE-binding mutant or a recombinant low IgE-binding mutant.

Allergens may be present in equi-molar amounts or the ratio of the allergens present may vary preferably up to 1:20.

In a further embodiment of the invention the low IgE binding allergen is an allergen according to WO 99/47680, WO 02/40676 or WO 03/096869.

Infectious Antigens

In a preferred embodiment of the invention, the microbial agent is a virus, a bacterium, a fungus, a parasite or any part thereof.

Examples of microbial agents are Vibrio species, Salmonella species, Bordetella species, Haemophilus species, Toxoplasmosis gondii, Cytomegalovirus, Chlamydia species, Streptococcal species, Norwalk Virus, Escherischia coli, Helicobacter pylori, Helicobacter felis, Rotavirus, Neisseria gonorrhae, Neisseria meningiditis, Adenovirus, Epstein Barr Virus, Japanese Encephalitis Virus, Pneumocystis carini, Herpes simplex, Clostridia species, Respiratory Syncytial Virus, Klebsielia species, Shigella species, Pseudomonas aeruginosa, Parvovirus, Campylobacter species, Rickettsia species, Varicella zoster, Yersinia species, Ross River Virus, J. C. Virus, Rhodococcus equi, Moraxella catarrhalis, Borrelia burgdorferi, Pasteurella haemolytica, poliovirus, influenza virus, Vibrio cholerae and Salmonella enterica serovar Typhi.

Further examples of microbial agents are those, which prevent or reduce the symptoms of the following diseases: Influenza, Tuberculosis, Meningitis, Hepatitis, Whooping Cough, Polio, Tetanus, Diphtheria, Malaria, Cholera, Herpes, Typhoid, HIV, AIDS, Measles, Lyme disease, Travellers Diarrhea, Hepatitis A, B and C, Otitis Media, Dengue Fever, Rabies, Parainfluenza, Rubella, Yellow Fever, Dysentery, Legionnaires Disease, Toxoplasmosis, Q-Fever, Haemorrhegic Fever, Argentina Haemorrhagic Fever, Caries, Chagas Disease, Urinary Tract Infection caused by E. coli, Pneumoccoccal Disease, Mumps, and Chikungunya.

Method of Evaluating the Effect of SAV on an Individual

The invention further relates to a method of evaluating the effect of Specific Allergy Vaccination (SAV) on an individual, the method comprising
a) subjecting the individual to SAV,
b) measuring the level of a biomarker antibody selected from the group consisting of IgA, IgG, IgE and IgX specific to the antigen in a biological sample from the individual, and
c) using the measurements obtained to evaluate the effect of the SAV.

The evaluation of the effect of SAV is carried out in the same manner as described in connection with the method of evaluating the therapeutic potential of a vaccination program.

Definitions

In connection with the present invention, the following expressions are used:

“Biological sample” means any body fluid, such as blood, plasma, serum, urine and saliva, which is excreted, secreted or transported within a biological organism.

The expression “allergy” means any type 1, 2, 3 or 4 hyper-sensitivity allergy towards an antigen.

The expression “digestive antigen” means any antigenic agent which comes into contact with the mucosa of the digestive system, in particular the mucosa of the oral cavity, the pharynx, the larynx, the stomach and the intestine.

The expression “respiratory antigen” means any antigenic agent which comes into contact with the mucosa of the respiratory system, in particular the mucosa of the nose, the oral cavity, the pharynx, the larynx, the trachea and the lungs.

The expression “antigen” means any antigen, to which an individual may be exposed, and it refers to any naturally occurring or synthetic compound or substance, or part or fraction thereof that has been reported or can be shown to induce an immune response upon exposure to an individual.

The expression “allergen” means any allergen, to which an individual may be exposed, and it refers to any naturally occurring protein or mixture of proteins that have been reported to induce allergic, i.e. IgE mediated reactions, upon repeated exposure to an individual. The allergen evaluated may be in the form of an allergen extract, a purified allergen, a modified allergen, a recombinant allergen, a recombinant mutant allergen, any allergen fragment above 10 amino acids or any combination thereof.

The term “fast dispersing dosage form” refers to dosage forms which disintegrate in less than about 90 seconds, preferably in less than 60 seconds, preferably in less than 30 seconds, more preferably in less than 20, even more preferably in less than 10 seconds in the oral cavity, even more preferred in less than 5, most preferably in less than about 2 seconds of being placed in the oral cavity.

The term “oromucosa” means the mucosa of the oral cavity and the pharynx of the patient.

The expression “mucosa of the respiratory system” means the mucosa of the nose, the oral cavity, the pharynx, the larynx, the trachea and the lungs.

The expression “mucosa of the digestive system” means the mucosa of the oral cavity, the pharynx, the larynx, the stomach and the intestine.

The expression “genital mucosa” means the vaginal and urinal mucosa.

The term “therapeutic potential” means capable of partly or wholly preventing or treating an antigen-mediated immunological disease, or capable of partly or wholly alleviating symptoms or inhibiting causes of symptoms of an antigen-mediated immunological disease.

The term “IgX” means a parameter expressing directly or indirectly the level of allergen specific non-IgE antibodies, such as IgG4, present in the biological sample, which can compete with IgE on the binding of the allergen. IgX may e.g. be the absolute IgX level or the ratio of IgE as measured in an immunoassay with competition (interference) from other components of the biological sample to the level of IgE as measured in an immunoassay with no competition.

### EXAMPLES

### Example 1

### Treatment of Grass Pollen Allergic Patients with Fast-Dispersing Non-Compressed Sublingual Tablets

Background

A vaccine against grass pollen allergy using an extract of Phleum pratense as allergenic active substance is known to be effective in a formulation for subcutaneous administration, wherein the allergen is formulated together with an aluminium hydroxide gel as adjuvant.

Purpose

To test the therapeutic potential (efficacy) of a new formulation of extract of Phleum pratense in the form of a fast-dispersing, non-compressed, freeze-dried tablet for sublingual administration, the tablet containing no adjuvant. The tablet contained fish gelatine as matrix forming agent. The efficacy study constitutes a part of a clinical phase I study, which also includes a safety study.

Vaccination Protocol

Test persons

48 adult test persons between 18 and 65 years suffering from moderate to severe allergic rhinoconjunctivitis in the grass pollen season and having no symptoms outside the season. Inclusion criteria were 1) the clinical history of the test person, 2) a positive Skin Prick Test (>3 mm in a Soluprick© SQ-U HEP Phleum pratense), and 3) positive specific IgE against Phleum pratense (CAP class 2 or higher).

Doses and Administration Program

The 48 test persons were divided into four groups A, B, C and D each comprising 12 persons. Group A (2,500 SQ-U) is treated with one tablet containing 2,500 SQ-U and two placebo tablets. Group B (25,000 SQ-U) is treated with one tablet containing 25,000 SQ-U and two placebo tablets. Group C (75,000 SQ-U) is treated with three tablets containing 25,000 SQ-U. Group D is treated with three placebo tablets. The three tablets are administered one time each day for 8 weeks. The 8 weeks of treatment were followed by 10 weeks of no treatment, and then the same treatment as carried out in the initial 8 weeks was continued in 15 weeks.

Prior to the efficacy testing 45 of the persons had participated in a safety study, wherein 30 persons had been given increasing doses of from 2500 to 1,125,000 SQ-U and 15 persons had been given placebo.

Measurements

Blood serum samples were obtained after 0, 3-5 and 7-8 weeks of the 8 week treatment period as well as at 2 weeks after the end of the 15 week treatment period. The level of IgE, IgA and IgG were measured using the assays outlined below.

Assay Procedure for Measuring IgE

IgE was measured on an an ADVIA Centaur (Bayer) using a two-site immunoassay with the following procedure:
(a) mixing the liquid sample with a class-specific antibody directed against the antibody to be detected bound to paramagnetic particles to form a first solid phase complex,
(b) washing,
(c) adding Phleum pratense allergen bound to biotin to form a second solid phase complex,
(d) adding a chemiluminescent acridinium compound bound to streptavidin to form a third solid phase complex,
(e) magnetically separating the solid phase from the liquid phase,
(f) washing,
(g) initiating a chemiluminescent reaction, if any, in the separated solid phase and
(h) measuring the chemiluminescense emitted, which is indicative of the presence of said antibody in the sample.
   Assay Procedure for Measuring IgA and IgG
1. Coating. 100 μl Phl p (10 μg/ml) extract is added to the wells of an ELISA plate (NUNC Maxisorp 439454). The plates are allowed to stand until the next day at 2-8° C.
2. Washing. The coated plates are washed with a buffer.
3. Blocking. 200 μl 2% Casein buffer is added to each well and incubated at room temperature for one hour on a shaking table. After incubation the Casein buffer is removed.
4. Serum. The serum sample is diluted, and 100 μl diluted sample is added to the well of a plate and incubated at room temperature for two hours on a shaking table.
5. Washing.
6. Conjugate. 100 μl HRP mouse anti-human IgG/IgA diluted 1:1000 in 0.5% Casein buffer is added to each well and allowed to stand at room temperature for one hour on a shaking table.
7. Washing.
8. Substrate: 100 μp TMP (3,3′,5,5′-Tetramethylbenzidine, Kem-En-Tec TMB ONE) is added to each well and incubated 20 min.
9. Stop. 100 μl 0.5 M H₂SO₄ is added to each well to stop the reaction.
10. Measurement. The resulting reaction mixtures are subjected to a spectrophotometric measurement at 450 nm endpoint (Bio Kinetics Reader EL-340).
   Results

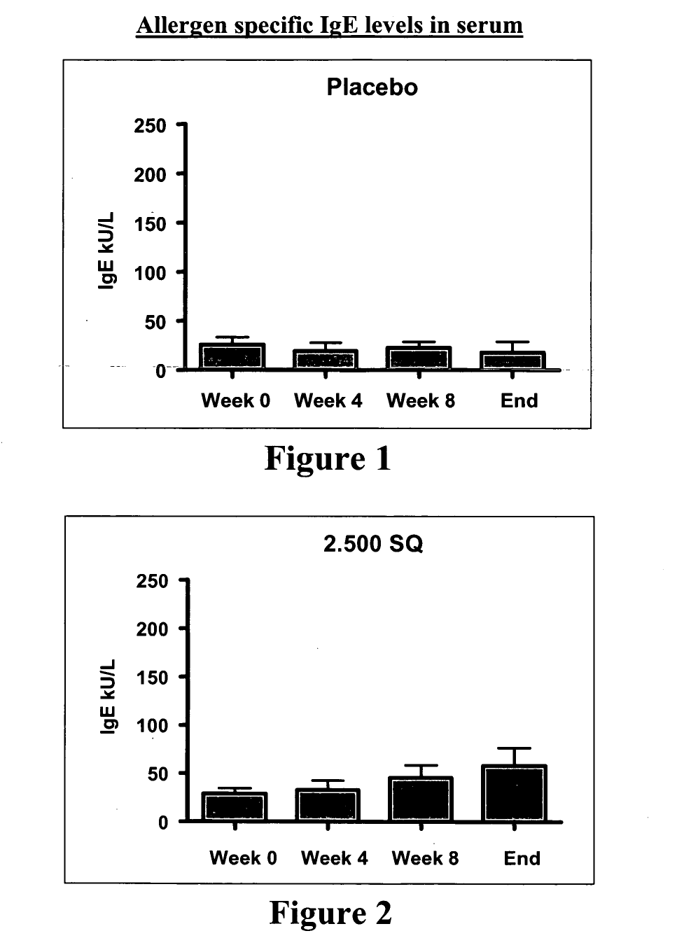

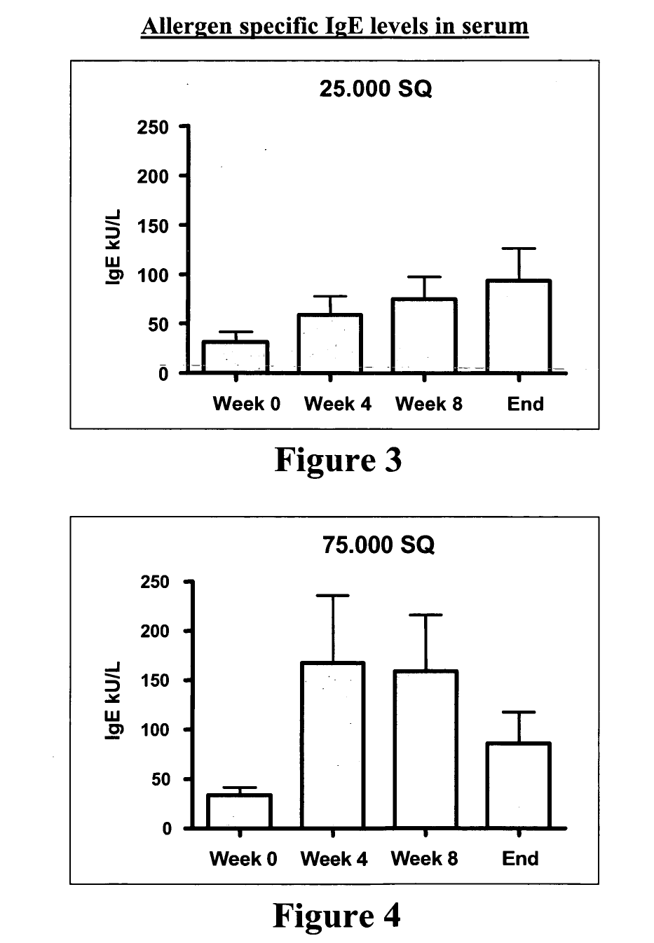

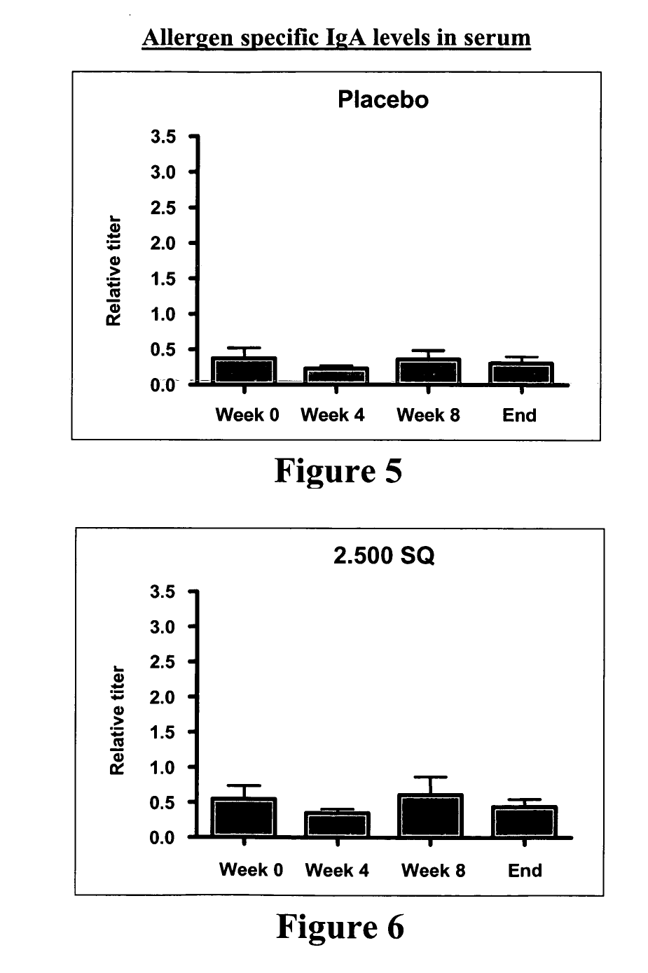

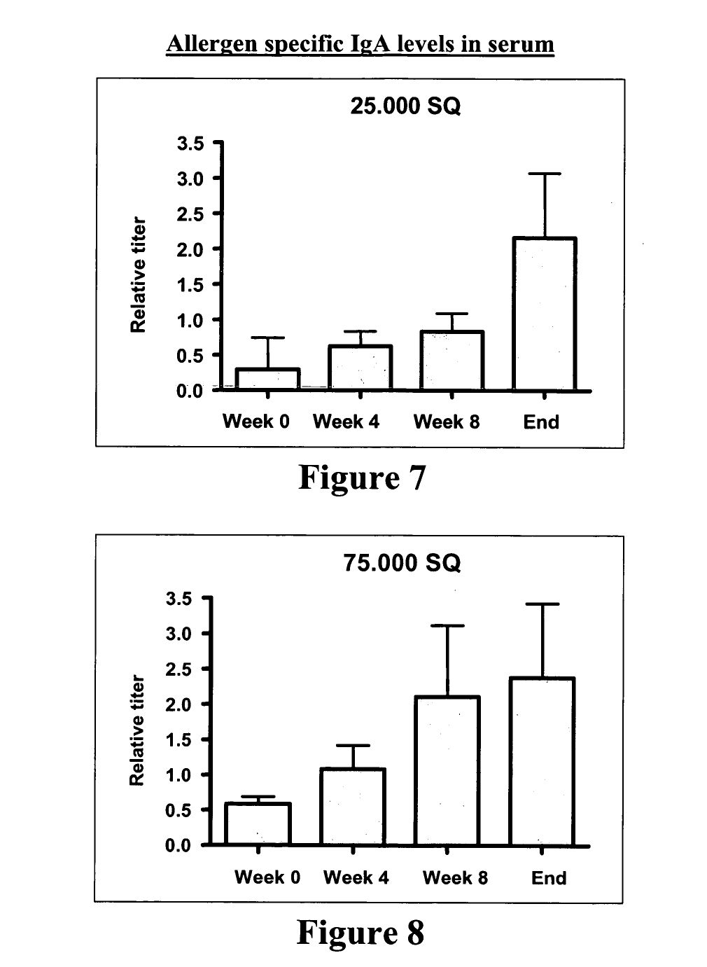

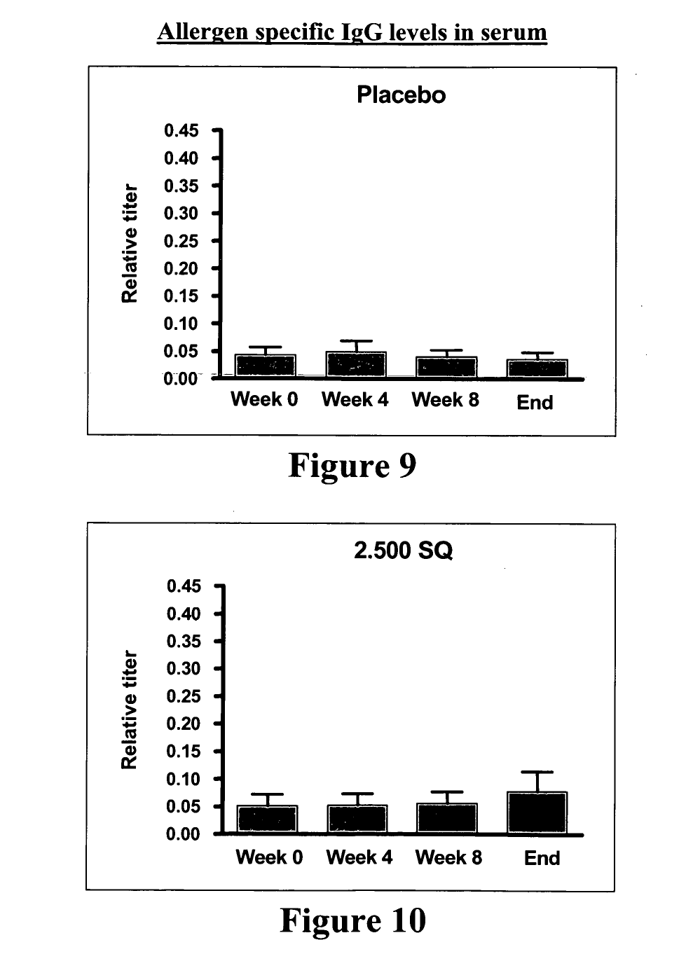

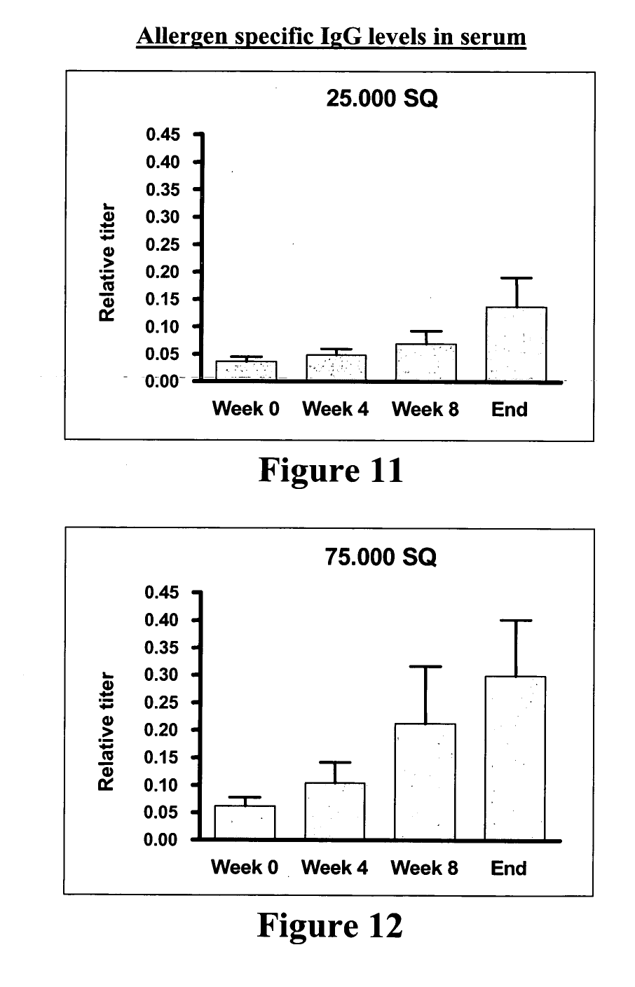

The results are shown in FIGS. 1-12, wherein FIG. 1-4 show the IgE levels for groups A, B, C and D, respectively, FIG. 5-8 show the IgA levels for groups A, B, C and D, respectively, and FIG. 9-12 show the IgG levels for groups A, B, C and D, respectively, and wherein each figure indicate the antibody level (kU/L for IgE and relative titer as compared to a reference serum from an allergic patient for IgA and IgG) for the four points of time of measurement (week 0, week 4 (samples obtained at 3-5 weeks), week 8 (samples obtained at 7-8 weeks) and End (two weeks after the end of the 15 week treatment period). The antibody level is mean values for the group treated.

From FIG. 1-4 the following appears: In treatment Group A (Placebo) no increase in the IgE level is registered over the period of administration. For treatment Group B (2500 SQ), a continuous increase in the IgE level from 29 to 58 is registered over the period. For treatment Group C (25000 SQ), an continuous increase from 31 to 94 is observed. Finally, for treatment Group D (75000 SQ), the IgE level raises sharply from 34 at 0 weeks to a maximum value of 168 at 4 weeks, and then falls to 159 at 8 weeks and further to 86 at End. The temporal profile for Group D is considered to represent a desired profile, which signifies an effective Specific Allergy Vaccination.

From FIG. 5-8 it appears that no increase in the IgA level is registered for treatment Groups A and B. For treatment Group C and D the level of IgA increases steadily over the period of administration.

From FIG. 9-12 it appears that no increase in the IgG level is registered for treatment Groups A and B. For treatment Group C and D the level of IgG increases steadily over the period of administration.

The results shown in FIG. 1-12 are further listed in Table 1.

TABLE 1

| Ab | N | Dose | Week 0 | Week 4 | Week 8 | End |
|---|---|---|---|---|---|---|
| IgE | 10 | Placebo | 25.5 | 18.8 | 22.4 | 17.5 |
| IgE | 12 | 2.500 SQ-U | 28.9 | 32.6 | 45.2 | 57.8 |
| IgE | 12 | 25.000 SQ-U | 31.1 | 58.9 | 74.9 | 93.6 |
| IgE | 11 | 75.000 SQ-U | 33.8 | 167.6 | 159.2 | 86.2 |
| IgA | | Placebo | 0.373 | 0.228 | 0.356 | 0.308 |
| IgA | | 2.500 SQ-U | 0.545 | 0.339 | 0.613 | 0.435 |
| IgA | | 25.000 SQ-U | 0.500 | 0.526 | 0.773 | 2.155 |
| IgA | | 75.000 SQ-U | 0.586 | 1.078 | 2.108 | 2.380 |
| IgG | | Placebo | 0.0432 | 0.049 | 0.0404 | 0.0356 |
| IgG | | 2.500 SQ-U | 0.05117 | 0.0523 | 0.05558 | 0.07763 |
| IgG | | 25.000 SQ-U | 0.036 | 0.04827 | 0.06833 | 0.1364 |
| IgG | | 75.000 SQ-U | 0.0615 | 0.1041 | 0.2124 | 0.2996 |

### Example 2

### Treatment of Grass Pollen Allergic Patients with Fast-Dispersing Non-Compressed Sublingual Tablets

Background

A vaccine against grass pollen allergy using an extract of Phleum pratense as allergenic active substance is known to be effective in a formulation for subcutaneous administration, wherein the allergen is formulated together with an aluminium hydroxide gel as adjuvant.

Purpose

To test the therapeutic potential (efficacy) of a new formulation of extract of Phleum pratense in the form of a fast-dispersing, non-compressed, freeze-dried tablet for sublingual administration, the tablet containing no adjuvant. The tablet contained fish gelatine as matrix forming agent. The efficacy study constitutes a part of a clinical phase I study, which also includes a safety study.

Vaccination Protocol

Test Persons

9 adult test persons between 18 and 65 years suffering from moderate to severe allergic rhinoconjunctivitis in the grass pollen season and having no symptoms outside the season. Inclusion criteria were 1) the clinical history of the test person, 2) a positive Skin Prick Test (>3 mm in a Soluprick® SQ-U HEP Phleum pratense), and 3) positive specific IgE against Phleum pratense (CAP class 2 or higher).

Doses and Administration Program

6 test persons were treated with a number of tablets corresponding to a daily dose of 1.000.000 SQ-U for a period of 28 days, and 3 persons were treated with placebo for the same period.

Measurements

Biological samples were obtained at the start of and at 1-2 weeks after the last day of treatment. The level of IgE was measured using the assay described in Example 1.

Results

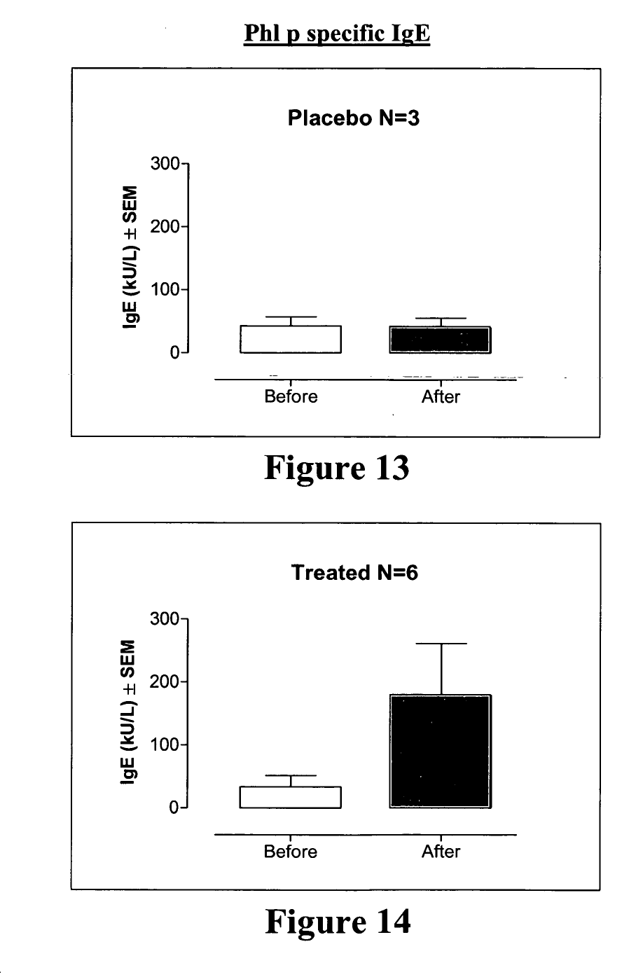

The results are shown in FIGS. 13-14, wherein FIG. 13 shows the IgE level for the group of patients treated with placebo at the start of and at 1-2 weeks after a treatment period, and wherein FIG. 14 shows the IgE level for the group of patients treated with a dose of 1000000 SQ at the start of and at 1-2 weeks after a treatment period.

As will appear from FIG. 13, the IgE level is unchanged during the treatment period for the placebo group, whereas the IgE level increases from about 0.31 to about 1.79.

### Example 3

### Treatment of Grass Pollen Allergic Patients With Fast-Dispersing Non-Compressed Sublingual Tablets

Background

A vaccine against grass pollen allergy using an extract of Phleum pratense as allergenic active substance is known to be effective in a formulation for subcutaneous administration, wherein the allergen is formulated together with an aluminium hydroxide gel as adjuvant.

Purpose

To test the therapeutic potential (efficacy) of a new formulation of extract of Phleum pratense in the form of a fast-dispersing, non-compressed, freeze-dried tablet for sublingual administration, the tablet containing no adjuvant.

The tablet contained fish gelatine as matrix forming agent. The efficacy study constitutes a part of a clinical phase IIb-III study.

Vaccination Protocol

Test Persons

855 adult test persons between 18 and 65 years suffering from moderate to severe allergic rhinoconjunctivitis in the grass pollen season and having no symptoms outside the season. Inclusion criteria were 1) the clinical history of the test person, 2) a positive Skin Prick Test (>3 mm in a Soluprick® SQ-U HEP Phleum pratense), and 3) positive specific IgE against Phleum pratense (CAP class 2 or higher). The test persons lived in Denmark, Germany, Sweden, Norway, Belgium, Austria, England, Switzerland and Canada.

Doses and Administration Program

The 855 test persons were divided into six groups 1, 2, 3, 4, 5 and 6 comprising 136, 136, 139, 141, 150 and 153 persons, respectively.
Group 1 was treated with placebo and active anti-histamine.
Group 2 was treated with an allergen dose of 2500 SQ and active anti-histamine.
Group 3 was treated with an allergen dose of 25000 SQ and active anti-histamine.
Group 4 was treated with an allergen dose of 75000 SQ and active anti-histamine.
Group 5 was treated with placebo and placebo anti-histamine.
Group 6 was treated with an allergen dose of 75000 SQ and placebo anti-histamine.

The treatment was carried out daily for approx. 8 weeks prior to the anticipated start of the Phleum pratense pollen season (pre-treatment period), through the pollen season and for 1 week after the pollen season (Post-treatment period). The duration of the pollen season varied from 12 days in Trondheim (Norway) to 86 days in Karlsruhe, Germany.

Measurements

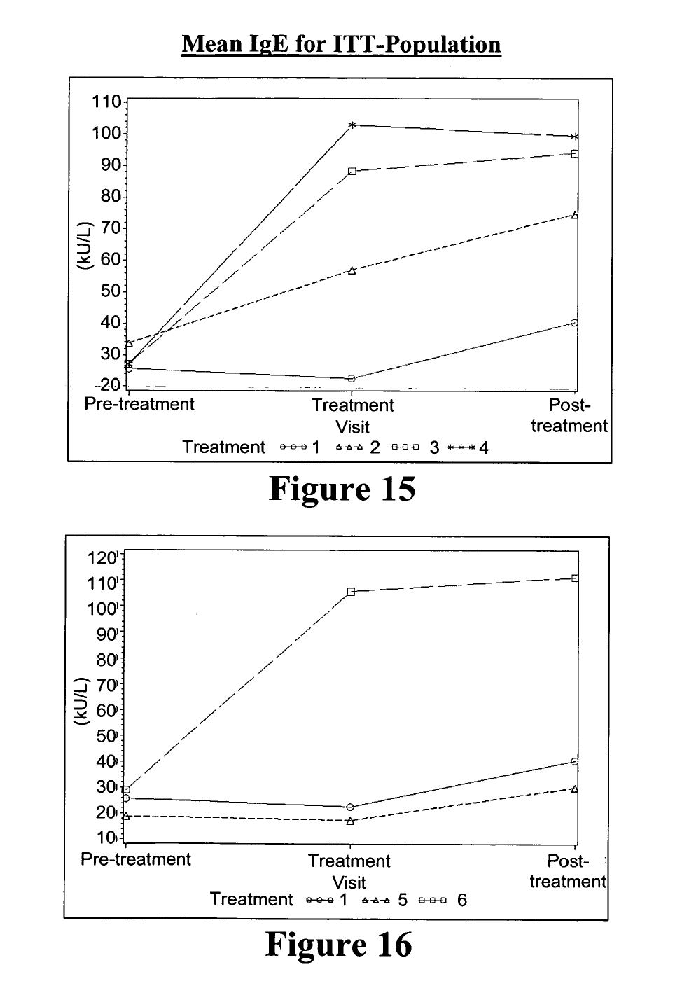

Blood samples were drawn at the start of the pre-treatment period (Pre-treatment in FIG. 15-22), at the start of the pollen season (Treatment in FIGS. 15-22) and at the end of the Post-treatment period (Post-treatment in FIGS. 15-22). The level of IgE was measured using the assay described in Example 1. The IgX parameter was determined as IgE Level (competition)/IgE Level (no competition). IgE Level (no competition) was measured using the assay described in Example 1. IgE Level (competition) was measured using the following assay procedure:
(a) mixing the liquid sample with a class-specific antibody directed against the antibody to be detected bound to paramagnetic particles to form a first solid phase complex,
(b) adding Phleum pratense allergen bound to biotin to form a second solid phase complex,
(c) adding a chemiluminescent acridinium compound bound to streptavidin to form a third solid phase complex,
(d) magnetically separating the solid phase from the liquid phase,
(e) washing
(f) initiating a chemiluminescent reaction, if any, in the separated solid phase and
(h) measuring the chemiluminescense emitted, which is indicative of the presence of said antibody in the sample.
   Results

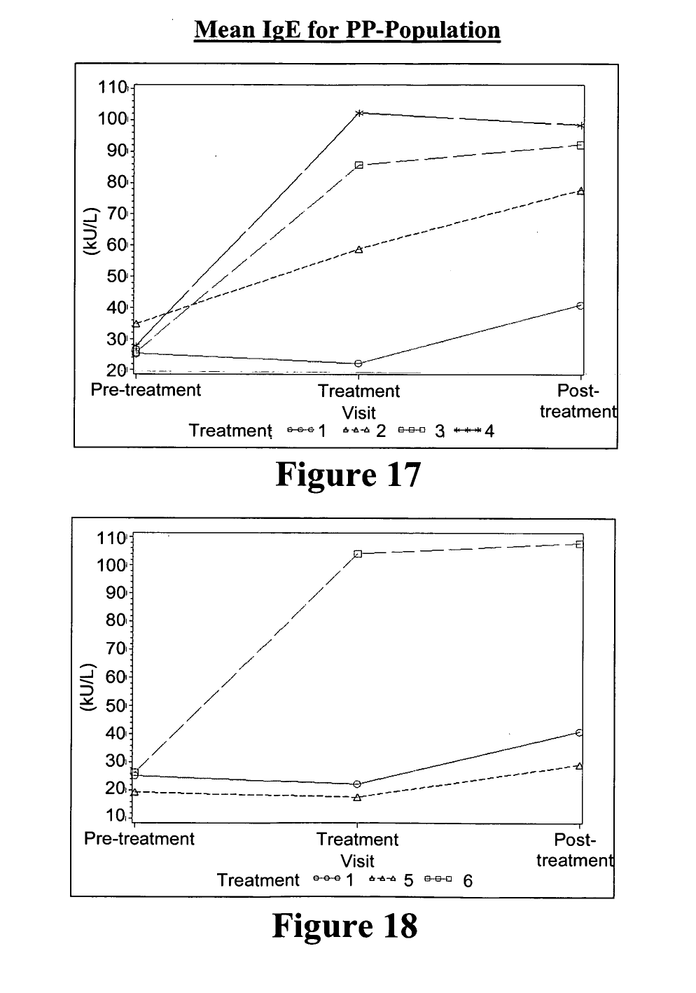

The IgE results are shown in FIGS. 15-18 and Table 2 and 3, wherein FIG. 15 shows the IgE level for Groups 1-4 for the ITT-population, FIG. 16 shows the IgE level for Groups 1, 5 and 6 for the ITT-population, FIG. 17 shows the IgE level for Groups 1-4 for the PP-population and FIG. 18 shows the IgE level for Groups 1, 5 and 6 for the PP-population. ITT means Intention To Treat and includes all persons starting out in the study, and PP means Per Protocol and includes all persons treated according to the planned protocol. Table 2 contains all values of FIGS. 15 and 16 and Table 3 contains all values of FIGS. 17 and 18.

As will appear from FIGS. 15-18 and Tables 2 and 3, for Groups 1 and 5 no increase in the IgE level from Pre-treatment to Treatment is registered, and a small increase in the IgE level from Treatment to Post-treatment is registered. For Group 2 a moderate steady increase in the IgE level is registered. For Groups 3, 4 and 6 a sharp increase in the IgE level from Pre-treatment to Treatment is registered followed by a plateau phase from Treatment to Post-treatment. For Group 4, the IgE level decreases slightly during the plateau phase. The temporal profile of Group 3, 4 and 6, in particular Group 4, is considered to represent a desired profile, which signifies an effective Specific Allergy Vaccination.

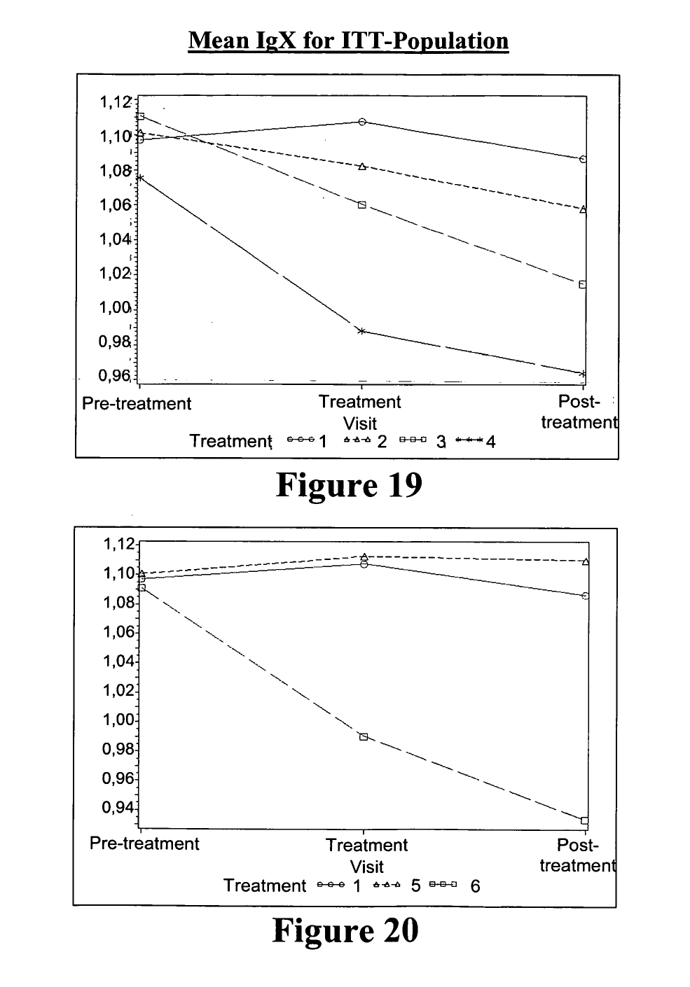

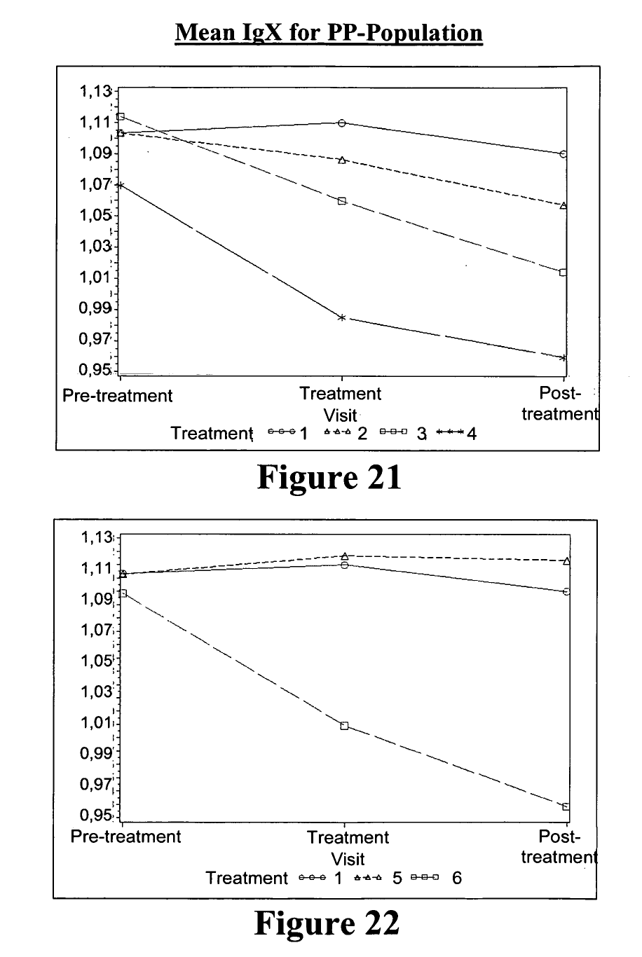

The IgX results are shown in FIGS. 19-22 and Table 4 and 5, wherein FIG. 19 shows the IgX level for Groups 1-4 for the ITT-population, FIG. 20 shows the IgX level for Groups 1, 5 and 6 for the ITT-population, FIG. 21 shows the IgX level for Groups 1-4 for the PP-population and FIG. 22 shows the IgX level for Groups 1, 5 and 6 for the PP-population. ITT means Intention To Treat and includes all persons starting out in the study, and PP means Per Protocol and includes all persons treated according to the planned protocol. Table 4 contains all values of FIGS. 19 and 20 and Table 5 contains all values of FIGS. 21 and 22.

As will appear from FIGS. 19-22 and Tables 4 and 5, for Groups 1 and 5 no decrease in the IgX level from Pre-treatment to Treatment is registered, and a small decrease in the IgX level from Treatment to Post-treatment is registered. For Groups 2 and 3 a moderate steady decrease in the IgX level is registered. For Groups 4 and 6 a strong decrease in the IgX level from Pre-treatment to Treatment is registered followed by a less strong decrease from Treatment to Post-treatment. For Group 4, most of the IgX level decrease occurs in the period from Pre-treatment to Treatment. The temporal profile of Groups 4 and 6, in particular Group 4, is considered to represent a desired profile, which signifies an effective Specific Allergy Vaccination.

TABLE 2
Grass specific IgE (kU/L) for ITT-Population

| | Group 1 | Group 2 | Group 3 | Group 4 | Group 5 | Group 6 | |
|---|---|---|---|---|---|---|---|
| | Placebo + | 2500SQ + | 25000SQ + | 75000SQ + | Placebo + | 75000SQ + | |
| | Active | Active | Active | Active | Placebo | Placebo | Overall |
| Visit | (N = 136) | (N = 136) | (N = 139) | (N = 141) | (N = 150) | (N = 153) | (N = 855) |
| Pre-treatment | | | | | | | |
| N | 136 | 135 | 139 | 139 | 148 | 153 | 850 |
| Mean(SD) | 25.7(34.4) | 3.8(47.2) | 27.2(34.6) | 27.1(45.8) | 18.8(28.0) | 29.1(44.4) | 26.9(39.8) |
| Median | 12.4 | 14.0 | 12.7 | 12.4 | 10.1 | 10.6 | 12.0 |
| P5%-P95% | 1.3-109.0 | 1.6-120.9 | 0.8-115.4 | 1.1-97.2 | 0.5-71.4 | 0.7-133.8 | 0.8-107.4 |
| Min-Max | 0.3-201.5 | 0.2-271.8 | 0.4-207.1 | 0.4-360.6 | 0.1-185.6 | 0.2-229.6 | 0.1-360.6 |
| Treatment | | | | | | | |
| N | 130 | 131 | 130 | 131 | 142 | 145 | 809 |
| Mean(SD) | 22.7(29.5) | 57.1(87.5) | 88.3(104.6) | 103.0(126.1) | 17.5(28.3) | 105.7(133.2) | 65.8(101.4) |
| Median | 11.2 | 24.9 | 46.4 | 63.4 | 9.3 | 57.0 | 26.6 |
| P5%-P95% | 0.9-92.8 | 1.6-173.1 | 1.9-326.8 | 3.0-355.5 | 0.4-63.5 | 2.7-340.2 | 1.2-258.8 |
| Min-Max | 0.2-148.8 | 0.2-702.1 | 0.5-563.3 | 1.1-705.6 | 0.0-233.4 | 0.5-901.7 | 0.0-901.7 |
| Post-treatment | | | | | | | |
| N | 130 | 131 | 130 | 133 | 142 | 146 | 812 |
| Mean(SD) | 40.7(53.1) | 74.8(96.1) | 94.1(107.5) | 99.5(129.4) | 30.2(46.7) | 111.1(136.7) | 75.2(105.6) |
| Median | 18.9 | 38.6 | 53.7 | 58.3 | 15.4 | 59.7 | 36.1 |
| P5%-P95% | 1.5-154.1 | 2.8-250.2 | 2.8-360.0 | 3.3-350.6 | 1.0-101.6 | 4.5-356.0 | 1.9-290.1 |
| Min-Max | 0.3-286.8 | 0.3-675.5 | 1.6-554.9 | 1.5-715.7 | 0.0-278.9 | 1.1-928.8 | 0.0-928.8 |

TABLE 3
Grass specific IgE (kU/L) for PP-Population

| | Group 1 | Group 2 | Group 3 | Group 4 | Group 5 | Group 6 | |
|---|---|---|---|---|---|---|---|
| | Placebo + | 2500SQ + | 25000SQ + | 75000SQ + | Placebo + | 75000SQ + | |
| | Active | Active | Active | Active | Placebo | Placebo | Overall |
| Visit | (N = 122) | (N = 122) | (N = 125) | (N = 124) | (N = 128) | (N = 127) | (N = 748) |
| Pre-treatment | | | | | | | |
| N | 122 | 121 | 125 | 122 | 127 | 127 | 744 |
| Mean(SD) | 25.3(35.0) | 34.7(48.6) | 25.7(34.5) | 27.7(48.1) | 19.4(28.7) | 26.4(40.3) | 26.5(39.9) |
| Median | 12.1 | 14.1 | 11.0 | 11.9 | 11.1 | 10.5 | 11.6 |
| P5%-P95% | 1.3-109.0 | 1.6-120.9 | 0.8-103.1 | 1.5-97.2 | 0.4-71.4 | 0.6-107.1 | 0.8-109.0 |
| Min-Max | 0.3-201.5 | 0.2-271.8 | 0.4-207.1 | 0.4-360.6 | 0.1-185.6 | 0.2-219.0 | 0.1-360.6 |
| Treatment | | | | | | | |
| N | 122 | 121 | 123 | 123 | 127 | 127 | 743 |
| Mean(SD) | 22.3(29.2) | 58.9(90.2) | 85.7(102.5) | 102.4(127.9) | 17.6(28.9) | 104.1(133.2) | 65.2(101.4) |
| Median | 11.2 | 25.0 | 44.9 | 63.3 | 9.3 | 57.0 | 26.7 |
| P5%-P95% | 0.9-92.8 | 1.7-173.1 | 1.9-280.4 | 3.4-355.5 | 0.4-63.5 | 2.7-327.4 | 1.3-251.3 |
| Min-Max | 0.2-148.8 | 0.2-702.1 | 0.5-563.3 | 1.1-705.6 | 0.0-233.4 | 0.5-901.7 | 0.0-901.7 |
| Post-treatment | | | | | | | |
| N | 122 | 121 | 121 | 123 | 127 | 126 | 740 |
| Mean(SD) | 40.9(54.3) | 77.8(98.8) | 92.2(105.7) | 98.4(131.2) | 29.2(44.4) | 107.7(136.5) | 74.3(105.3) |
| Median | 18.5 | 38.6 | 53.2 | 57.8 | 15.7 | 53.2 | 34.9 |
| P5%-P95% | 1.5-154.1 | 3.2-250.2 | 2.8-324.7 | 3.3-350.6 | 0.7-94.0 | 3.1-356.0 | 2.0-288.4 |
| Min-Max | 0.3-286.8 | 0.5-675.5 | 1.6-554.9 | 1.5-715.7 | 0.0-278.9 | 1.1-928.8 | 0.0-928.8 |

TABLE 4
Grass specific IgX for ITT-Population

| | Group 1 | Group 2 | Group 3 | Group 4 | Group 5 | Group 6 | |
|---|---|---|---|---|---|---|---|
| | Placebo + | 2,500 SQ + | 25,000 SQ + | 75,000 SQ + | Placebo + | 75,000 SQ + | |
| | Active | Active | Active | Active | Placebo | Placebo | Overall |
| Visit | (N = 136) | (N = 136) | (N = 139) | (N = 141) | (N = 150) | (N = 153) | (N = 855) |
| Pre-treatment | | | | | | | |
| N | 136 | 135 | 139 | 139 | 148 | 153 | 850 |
| Mean (SD) | 1.097(0.15) | 1.101(0.15) | 1.111(0.16) | 1.075(0.13) | 1.101(0.21) | 1.091(0.18) | 1.096(0.16) |
| Median | 1.08 | 1.08 | 1.09 | 1.07 | 1.07 | 1.08 | 1.08 |
| P5%-P95% | 0.88-1.37 | 0.90-1.37 | 0.90-1.42 | 0.87-1.30 | 0.87-1.44 | 0.85-1.39 | 0.88-1.38 |
| Min-Max | 0.80-1.75 | 0.81-1.64 | 0.66-1.78 | 0.79-1.44 | 0.67-2.68 | 0.57-1.69 | 0.57-2.68 |
| Treatment | | | | | | | |
| N | 130 | 131 | 130 | 131 | 142 | 145 | 809 |
| Mean (SD) | 1.107(0.16) | 1.082(0.14) | 1.060(0.17) | 0.988(0.13) | 1.113(0.25) | 0.990(0.16) | 1.056(0.18) |
| Median | 1.09 | 1.06 | 1.02 | 1.00 | 1.07 | 0.97 | 1.03 |
| P5%-P95% | 0.88-1.41 | 0.90-1.35 | 0.88-1.46 | 0.80-1.20 | 0.86-1.46 | 0.77-1.25 | 0.83-1.35 |
| Min-Max | 0.77-1.76 | 0.83-1.53 | 0.64-1.76 | 0.55-1.39 | 0.79-3.19 | 0.47-1.48 | 0.47-3.19 |
| Post-treatment | | | | | | | |
| N | 130 | 131 | 130 | 133 | 142 | 146 | 812 |
| Mean (SD) | 1.087(0.16) | 1.058(0.14) | 1.015(0.16) | 0.964(0.16) | 1.110(0.27) | 0.934(0.17) | 1.027(0.19) |
| Median | 1.07 | 1.03 | 0.99 | 0.95 | 1.07 | 0.95 | 1.02 |
| P5%-P95% | 0.87-1.35 | 0.88-1.33 | 0.81-1.38 | 0.71-1.20 | 0.87-1.44 | 0.67-1.19 | 0.78-1.32 |
| Min-Max | 0.76-1.65 | 0.70-1.62 | 0.56-1.64 | 0.53-1.65 | 0.80-3.48 | 0.29-1.24 | 0.29-3.48 |

TABLE 5
Grass specific IgX for PP-Population

| | Group 1 | Group 2 | Group 3 | Group 4 | Group 5 | Group 6 | |
|---|---|---|---|---|---|---|---|
| | Placebo + | 2,500 SQ + | 25,000 SQ + | 75,000 SQ + | Placebo + | 75,000 SQ + | |
| | Active | Active | Active | Active | Placebo | Placebo | Overall |
| Visit | (N = 122) | (N = 122) | (N = 125) | (N = 124) | (N = 128) | (N = 127) | (N = 748) |
| Pre-treatment | | | | | | | |
| N | 122 | 121 | 125 | 122 | 127 | 127 | 744 |
| Mean (SD) | 1.103(0.15) | 1.103(0.15) | 1.114(0.17) | 1.070(0.12) | 1.103(0.22) | 1.088(0.18) | 1.097(0.17) |
| Median | 1.08 | 1.10 | 1.09 | 1.07 | 1.07 | 1.07 | 1.08 |
| P5%-P95% | 0.89-1.37 | 0.90-1.37 | 0.91-1.42 | 0.88-1.29 | 0.87-1.44 | 0.84-1.40 | 0.88-1.39 |
| Min-Max | 0.80-1.75 | 0.81-1.64 | 0.66-1.78 | 0.79-1.44 | 0.67-2.68 | 0.57-1.69 | 0.57-2.68 |
| Treatment | | | | | | | |
| N | 122 | 121 | 123 | 123 | 127 | 127 | 743 |
| Mean(SD) | 1.110(0.16) | 1.086(0.13) | 1.060(0.17) | 0.985(0.13) | 1.117(0.25) | 0.989(0.16) | 1.058(0.18) |
| Median | 1.09 | 1.06 | 1.02 | 1.00 | 1.07 | 0.97 | 1.04 |
| P5%-P95% | 0.88-1.41 | 0.90-1.35 | 0.88-1.46 | 0.80-1.20 | 0.86-1.40 | 0.77-1.25 | 0.83-1.35 |
| Min-Max | 0.77-1.76 | 0.83-1.53 | 0.64-1.76 | 0.55-1.39 | 0.81-3.19 | 0.47-1.48 | 0.47-3.19 |
| Post-treatment | | | | | | | |
| N | 122 | 121 | 121 | 123 | 127 | 126 | 740 |
| Mean(SD) | 1.090(0.16) | 1.057(0.13) | 1.014(0.17) | 0.960(0.16) | 1.113(0.28) | 0.929(0.17) | 1.027(0.20) |
| Median | 1.07 | 1.04 | 0.99 | 0.94 | 1.07 | 0.95 | 1.02 |
| P5%-P95% | 0.88-1.35 | 0.88-1.32 | 0.81-1.38 | 0.71-1.20 | 0.87-1.44 | 0.65-1.19 | 0.79-1.32 |
| Min-Max | 0.76-1.65 | 0.70-1.62 | 0.56-1.64 | 0.53-1.65 | 0.80-3.48 | 0.29-1.24 | 0.29-3.48 |

1. A method of evaluating the therapeutic potential of a vaccination program comprising a vaccine for mucosal administration comprising one or more antigens and a vaccination protocol, the method comprising
a) subjecting at least one test individual to the vaccination program,
b) measuring the level of a biomarker antibody selected from the group consisting of IgA, IgG, IgE and IgX specific to the antigen in a biological sample from the test individual, and
c) using the measurements obtained to evaluate the therapeutic potential of the vaccination program.

2. A method according to claim 1, wherein the evaluation of the measurements obtained is carried out by comparison with a reference temporal biomarker antibody level profile.

3. A method according to claim 1, wherein the biomarker antibody is IgE.

4. A method according to claim 2, wherein the IgE level increases more 50%, preferably more than 100%, more preferably more than 200%, more preferably more than 300% and more preferably more than 400% compared to the level at the start of the vaccination program.

5. A method according to claim 4, wherein the IgE level increase occurs within twelve weeks, preferably within ten weeks, more preferably within eight weeks, preferably within four weeks from the start of the vaccination program.

6. A method according to claim 2, wherein the IgE level has a maximum value followed by a decrease.

7. A method according to claim 6, wherein the maximum value of the IgE level occurs within twelve weeks, preferably within ten weeks, more preferably within eight weeks, and more preferably within four weeks from the start of the vaccination program.

8. A method according to claim 6, wherein the IgE level decreases to a level of below 90%, preferably below 80%, more preferably below 70%, more preferably below 60%, and most preferably below 50% of the maximum value.

9. A method according to claim 8, wherein the IgE level decrease occurs within 26 weeks, preferably within 20 weeks, more preferably within 16 weeks, more preferably within 12 weeks, more preferably within 8 weeks and most preferably within 4 weeks from the time of the maximum value.

10. A method according to claim 1, wherein the biomarker antibody is IgA.

11. A method according to claim 10, wherein the IgA level increases more than 50%, preferably more than 100%, more preferably more than 200%, more preferably more than 300% and most preferably more than 400% compared to the level at the start of the vaccination program.

12. A method according to claim 11, wherein the IgA level increase occurs within 20 weeks, preferably within 16 weeks, more preferably within 12 weeks and most preferably within 8 weeks from the start of administration.

13. A method according to claim 1 to, wherein the biomarker antibody is IgG.

14. A method according to claim 13, wherein the IgG level increases more than 50%, preferably within 100%, more preferably more than 200%, more preferably more than 300% and most preferably more than 400% compared to the level at the start of the vaccination program.

15. A method according to claim 14, wherein the IgG level increase occurs within 20 weeks, preferably within 16 weeks, more preferably within 12 weeks and most preferably within 8 weeks from the start of administration.

16. A method according to claim 1, wherein the biomarker antibody is IgX.

17. A method according to claim 16, wherein IgX is expressed as the ratio of the level of IgE as measured in an immunoassay with competition from other components of the biological sample to the level of IgE as measured in an immunoassay with no competition.

18. A method according to claim 17, wherein the level of IgX decreases more than 4%, preferably more than 6%, more preferably more than 8%, more preferably more than 10%, more preferably more than 12% and most preferably more than 14% compared to the level at the start of the vaccination program.

19. A method according to claim 18, wherein the IgX level decrease occurs within 24 weeks, preferably within 16 weeks, more preferably within 12 weeks and most preferably within 8 weeks from the start of administration.

20. A method according to claim 1, wherein the vaccination protocol comprises administration of the vaccine to the test individual every day, every second day, every third day or every fourth day.

21. A method according to claim 20, wherein the vaccination protocol comprises administration of the vaccine for a period of more than 4 weeks, preferably more than 8 weeks, more preferably more than 12 weeks, more preferably more than 16 weeks, more preferably more than 20 weeks, more preferably more than 24 weeks, more preferably more than 30 weeks and most preferably more than 36 weeks.

22. A method according to claim 21, wherein the period of administration is a continuous period.

23. A method according to claim 21, wherein the period of administration is a discontinuous period interrupted by one or more periods of non-administration.

24. A method according to claim 23, wherein the period of non-administration is shorter than the period of administration.

25. A method according to claim 20, wherein the vaccine is administered to the test individual once a day.

26. A method according to claim 20, wherein the vaccine is administered to the test individual twice a day.

27. A method according to claim 20, wherein the vaccine is a uni-dose vaccine.

28. A method according to claim 1, wherein the vaccine is selected from the group consisting of vaccines formulated so as to be adapted to administration via the oromucosa, the mucosa of the respiratory system, the mucosa of the digestive system, the rectal mucosa and the genital mucosa.

29. A method according to claim 28, wherein the vaccine is formulated so as to be adapted to administration via the oromucosa.

30. A method according to claim 1, wherein the vaccine is selected from the group consisting of vaccines formulated as a solution, a suspension, fast dispersing dosage forms, drops and lozenges.

31. A method according to claim 30, wherein the vaccine is formulated as a fast dispersing dosage form.

32. A method according to claim 1, wherein the antigen is selected from the group consisting of a respiratory antigen, a digestive antigen, a microbial antigen and an insect antigen.

33. A method according to claim 32, wherein the antigen is a respiratory antigen.

34. A method according to claim 32, wherein the antigen is an allergen.

35. A method according to claim 34, wherein the allergen is an inhalant allergen.

36. A method according to claim 1, wherein the measuring of the level of biomarker antibody IgA, IgG and/or IgE specific to the antigen in a biological sample is carried out by an ELISA comprising the steps of 1) coating the allergen onto an Elisa plate and washing, 2) adding the biological sample, incubating and washing, 3) adding a conjugate of an enzyme and anti-biomarker antibody, incubating and washing, 4) adding an enzyme substrate, incubating and stopping the reaction, and 5) measuring the level of reacted substrate.

37. A method according to claim 1, wherein the measuring of the level of IgA, IgE and/or IgG specific to the antigen in a biological sample is carried out by a two-site immunoassay comprising the steps of
(a) mixing (i) the biological sample with (ii) a class-specific antibody directed against the antibody to be detected bound to a solid phase to form a mixture of a liquid phase and a two-component solid phase complex,
(b) separating the two-component solid phase complex from the liquid phase and washing the separated two-component solid phase complex to remove non-complex bound compounds,
(c) adding (iii) a ligand in the form of an antigen, an antibody or a hapten, and (iv) a label compound, to form a four-component solid phase complex,
(d) separating the four-component solid phase complex from the liquid phase,
(e) washing the separated four-component solid phase to remove non-complex bound compounds,
(f) performing a detection/measurement of the washed labelled four-component complex.

38. A method according to claim 37, wherein the two-site immunoassay comprises a two-site immunoassay for an antibody using a chemiluminescent label and a biotin bound ligand, said method comprising the steps of
(a) mixing (i) the biological sample with (ii) a class-specific antibody directed against the antibody to be detected bound to paramagnetic particles to form a mixture of a liquid phase and a two-component solid phase complex,
(b) magnetically separating the two-component solid phase complex from the liquid phase and washing the separated two-component solid phase complex to remove non-complex bound compounds,
(c) adding (iii) a ligand in the form of an antigen, an antibody or a hapten bound to biotin or a functional derivative thereof, and (iv) a chemiluminescent acridinium compound bound to avidin, streptavidin or a functional derivative thereof, to form a four-component solid phase complex,
(d) magnetically separating the four-component solid phase complex from the liquid phase,
(e) washing the separated four-component solid phase to remove non-complex bound compounds,
(f) initiating a chemiluminescent reaction, if any, in the washed solid phase, and detecting/measuring the resulting chemiluminescence, if any.

39. A method according to claim 1, wherein IgX is determined as the ratio of the level of IgE as measured in an immunoassay with competition from other components of the biological sample to the level of IgE as measured in an immunoassay with no competition.

40. A method according to claim 39, wherein IgE as measured in an immunoassay with competition is measured in an immunoassay comprising the steps of:
(a) mixing the biological sample with a ligand antigen, antibody or hapten bound to biotin or a functional derivative thereof, a class-specific antibody directed against the antibody to be detected bound to paramagnetic particles and a chemiluminescent acridinium compound bound to avidin, streptavidin or a functional derivative thereof to form a four-component solid phase complex,
(b) magnetically separating the four-component solid phase from the liquid phase,
(c) washing the separated four-component solid phase to remove non-complex bound compounds,
(d) initiating a chemiluminescent reaction, if any, in the separated solid phase and detecting/measuring the resulting chemiluminescence, if any.

41. A method according to claim 39, wherein IgE as measured in an immunoassay with no competition is measured in an immunoassay comprising the steps of:
(a) mixing (i) the biological sample with (ii) a class-specific antibody directed against the antibody to be detected bound to paramagnetic particles to form a mixture of a liquid phase and a two-component solid phase complex,
(b) magnetically separating the two-component solid phase complex from the liquid phase and washing the separated two-component solid phase complex to remove non-complex bound compounds,
(c) adding (iii) a ligand in the form of an antigen, an antibody or a hapten bound to biotin or a functional derivative thereof, and (iv) a chemiluminescent acridinium compound bound to avidin, streptavidin or a functional derivative thereof, to form a four-component solid phase complex,
(d) magnetically separating the four-component solid phase complex from the liquid phase,
(e) washing the separated four-component solid phase to remove non-complex bound compounds,
(f) initiating a chemiluminescent reaction, if any, in the washed solid phase, and detecting/measuring the resulting chemiluminescence, if any.

42. A method according to claim 1, wherein the biological sample is selected from the group consisting of blood, plasma, serum, urine, saliva and nasal secretion.

43. vaccine obtainable by the method according to claim 1.

44. A method of evaluating the effect of mucosal Specific Allergy Vaccination (SAV) on an individual, the method comprising
a) subjecting the individual to mucosal SAV,
b) measuring the level of a biomarker antibody selected from the group consisting of IgA, IgG, IgE and IgX specific to the antigen in a biological sample from the individual, and
c) using the measurements obtained to evaluate the effect of the SAV.
